(19) United States
(12) Patent Application Publication
(10) Pub. No.: US 2016/0287647 A1
(43) Pub. Date: Oct. 6, 2016

(54) METHODS FOR IMPROVEMENT IN SEXUAL HEALTH AND THE COMPOSITIONS USED THEREIN

(71) Applicant: OmniActive Health Technologies (Canada) Limited, Charlottetown (CA)

(72) Inventors: Jayant DESHPANDE, Charlottetown (CA); Khadija GHANAM, Charlottetown (CA); Vandita SRIVASTAVA, Pune (IN)

(21) Appl. No.: 15/087,233

(22) Filed: Mar. 31, 2016

(30) Foreign Application Priority Data
Mar. 31, 2015 (IN) 1313/MUM/2015

(51) Int. Cl. A61K 36/03; A61K 36/04; A61K 31/201; A61K 31/20; A61K 31/202

(52) U.S. Cl. CPC A61K 36/03; A61K 31/20; A61K 31/202; A61K 31/201; A61K 36/04; A61K 2236/39

(57) ABSTRACT

Methods are described for improving sexual health by administering a composition in an effective amount to a subject in need thereof. More particularly, methods are described for improving sexual health by administering a macroalgae composition including a macroalgae extract, comprised of saturated and unsaturated fatty acids in a ratio of at or about 1:0.5 to at or about 1:10. The composition is prepared by solvent extraction of macroalgae selected from green, red and/or brown seaweeds or the combinations thereof. Effects are evaluated on biomarkers related to sexual health such as testosterone synthesis, nitric oxide synthesis, phosphodiesterase 5 and 11.

### FIELD

Improvement in sexual health is described by administering compositions in an effective amount to a subject in need thereof. More particularly, methods are described for improving sexual health by administering a macroalgae composition including a macroalgae extract, which is comprised of saturated and unsaturated fatty acids in ratio, for example an area percentage ratio, of at or about 1:0.5 to at or about 1:10. The composition is prepared by solvent extraction of macroalgae selected from green, red, and/or brown seaweeds or the combinations thereof. The method as described herein is comprised of administering macroalgae composition to the subject in need thereof in an effective daily dose for improvement in sexual health. The method exerts the effect on biomarkers related to sexual health such as testosterone and nitric oxide synthesis, wherein both these biomarkers are enhanced by administering macroalgae composition in an effective amount to the subjects in need thereof. The method, as described herein also exerts effect on biomarkers such as phosphodiesterase 5 and 11, wherein both these biomarkers are inhibited, due to administration of macroalgae composition in an effective amount. The method for improvement in sexual health employs macroalgae composition which is comprised of saturated and unsaturated fatty acids in a specific ratio and is safe for human consumption, which can be administered in an effective amount to the subject in need thereof.

### BACKGROUND

The World Health Organization defines sexual health as a state of physical, emotional, mental and social well-being in relation to sexuality; it is not merely the absence of disease, dysfunction or infirmity. Sexual health is important part of physical health, mental and emotional health. Achieving sexual health allows for healthy relationships, planned pregnancies and disease prevention. That is why it is helpful to be well-informed about various aspects of sexual health. Similarly, it is helpful to be aware of factors that can complicate the sexual health. Age, physical changes, illness, disabilities and some medicines can affect sexual health in both men and women.

Sexual health is also known to be an important stress buster as known and proved by medical science, irrespective of age, civil status, or sexual orientation. Sexual problems in men and/or women are very common and impact overall health of entire society, thus affecting a person's well-being. It is well described in the scientific and medical literature that subjects who have low levels of sex hormones are more likely to be depressed than those with normal hormonal levels. They also suffer from the problems of virility and erectile dysfunction (ED). Testosterone occurs in two distinct forms in the body: free and bound. Free testosterone is useful and functional; bound testosterone is less so. As levels of free testosterone dwindle and estrogen levels increase, many men experience diminishing energy levels, decreasing muscle mass, deteriorating mood, abdominal obesity, and other problems related to sexual health.

Similarly many women face sexual health problems either in young age due to stress or medical conditions like hypothyroid or when they reach menopause and their ovaries produce smaller amounts of sex hormones. Lower levels of estrogen and less androgen leads to less sexual desire and the affected sexual health which has negative influence on their personality, family life and the overall performance at home and work front. Even though some treatment options like Viagra® and nitrate medications are available, there are possible undesired side effects, such as dizziness, fainting and even heart attack or stroke. Testosterone replacement drug therapy has gained enormous popularity in recent years. Further there are hundreds of products in the marketplace for virility and erectile dysfunction (ED). But most supplements purported to alleviate human sexual problems are marketed with little scientific evidence and instead rely on popular belief and personal testimonials. Companies are increasingly investing towards the discovery of new ingredients that fall primarily into 3 main categories: testosterone boosters, PDE-5 inhibitors (e.g. sildenafil-like activity), and stimulators of nitric oxide (NO) levels. Substances that elevate testosterone are popular not only in the sexual health market but also in the sports nutrition markets.

Alternatively, natural methods of boosting free testosterone levels and counteracting negative aspects of male aging have been in use for centuries. Specialized botanical extracts may help support healthy free testosterone levels, while reducing excess estrogen levels with age. A number of sexual health ingredients act on the endothelial nitric oxide (NO) synthase system.

Thus arginine is a precursor of NO whereas Panax ginseng has been shown to increase the production of NO in laboratory studies. Icariin, a flavanol glycoside compound, from Epimedium brevicornum (horny goat weed) is an example of a natural substance with PDE-5 inhibitory activity. Plant extracts such as chrysin and certain plant lignans inhibit the aromatization (conversion) of testosterone to estrogen, effectively enhancing free testosterone levels. Nettle root liberates testosterone in the body by preventing it from being bound to sex hormone-binding globulin. Lignans have also shown promising results against prostate cancer, while plants like muira puama have been shown to improve sexual health.

Yet there are a few natural products where some degree of scientific support exists suggesting a therapeutic benefit in ED. These include arginine, Eurycoma longifolia (tongkatali), Fenugreek, Ginkgo biloba, maca, Panax ginseng, Tribulus terrestris and yohimbine. These substances are oftenformulated in combination and with other ingredients, as products for sexual enhancement and to treat symptoms of andropause. However the results obtained from such products are not consistent as these do not take care of all physiological factors affecting sexual health.

As such, considering the foregoing, it may be appreciated that there continues to be a need for improved methods and compositions for enhancement of the human sexual health, without provoking negative physiological responses in the body. It would be desirable to have treatment options obtained preferably from natural source, which would be safe for human consumption and provide consistent and reliable results.

Macroalgae or seaweeds are known for their high nutritional values and they are rich in fibers, vitamins and proteins. They are also known to provide a carbohydrate rich diet and are used in soups, salads, and in confectionary items such as jellies and puddings. They are easily available along the sea shores and are known for many health applications.

There are references which describe process for the extraction of macroalgae or seaweeds and its various applications.

WO2015004255A1 relates to a process for the isolation of unique anti-oxidative glycoproteins from the pH precipitated fractions of enzymatic extracts of brown algae or brown seaweeds namely, Fucus serratus and Fucus vesiculosus which were hydrolysed by using 3 enzymes Alcalase, Viscozyme and Termamyl and the glycoproteins were isolated from these enzyme extracts.

WO2011057406A1 relates to protein concentrates and protein isolates comprising combinations of proteins, peptides and amino acids, as well as processes for their production. In particular, the disclosure relates to a process for removing fiber from macroalgae and/or microalgae to produce edible protein products.

US20120189706 relates to products derived from the exudate of kelp as well as high-purity Fucoidan derived from harvested kelp, specifically the brown algae Macrocystis pyrifera. More particularly, the reference relates to brown algae exudate and Macrocystis pyrifera derived pharmaceutical, nutraceutical, and cosmeceutical products and additives for oral, topical, and transdermal delivery to treat, prevent, or aid the health and wellness of an individual as well as the use of brown algae exudate and Macrocystis pyrifera high-purity Fucoidan as antioxidant additives for use with or in food, beverage, desert, or cosmetic products or in combination with other nutraceutical and cosmeceutical products to boost their final antioxidant impact.

WO2012089615A1 relates to a method of processing macroalgae matter to provide a nutraceutical preparation, the process comprises: a) heating a body of liquid containing the macroalgae matter during a first time period, b) collecting leached liquid from the macroalgae matter during the first time period, c) applying a hot liquid extraction step to the macroalgae matter during a second time period resulting in a liquid extract, the second time period occurring after the first time period has elapsed, and d) combining the leached liquid with the liquid extract.

US20080280994A1 relates to hot water extract of Ascophyllum, comprising at least about 20% by weight of polyphenolic compounds. Prior to aqueous extraction, lipophilic components are removed from the Ascophyllum by using hexane solvent and the remaining part is extracted with hot water. The aqueous extract is useful for inhibiting alpha-glucosidase activity; preventing or treating conditions mediated by alpha-glucosidase activity; reducing blood glucose levels; preventing or treating diabetes; modulating glucose uptake in adipocytes; preventing or treating obesity; scavenging free radicals; stimulating the immune system; activating macrophages; preventing or treating condition mediated by macrophage activation; and modulating nitric oxide production by macrophages.

WO2014083141A1 relates to an aqueous extraction process of active ingredients from the algae Ascophyllum nodosum for obtaining a stable extract of Ascophyllum nodosum. Further the stable aqueous extract may comprise ethanol up to a maximum of 20% in volume, and sulphated polysaccharides with high molecular weight.

US20150190950A1 relates to a method for preparing a powder from brown macroalgae, comprises treating the brown macroalgae with weak acids, followed by filtration, grinding and then drying the residue so as to obtain a powder with a particle size of between 0.5 and 1.5 mm wherein said macroalgae is chosen from among brown algae from the laminariales or fucales order.

WO2014078300A1 relates to a cosmetic active ingredient for improving the appearance of the skin beneath the eyes comprising an aqueous extract of Fucus vesiculosus.

US20070036821A1 relates to a method for increasing anti-thrombotic activity in a mammal comprising the step of administering to a mammal an amount of seaweed extract, prepared using water as solvent, in effective doses; wherein the seaweed is selected from the group consisting of Fucus vesiculosus, Fucus evanescens, Laminaria brasiliensis or Ascophylum nodosum.

US20050196410A1 relates to a method for retardation of inflammation in a mammal comprising the step of administering to a mammal an amount of aqueous seaweed extract in effective doses; wherein the seaweed is selected from the group consisting of Fucus vesiculosus, Fucus evanescens, Laminaria brasiliensisor Ascophylum nodosum.

### SUMMARY

The references are related to uses of macroalgae composition, which are prepared by employing solvents such as water and its application as biofuels, for the treatment of diabetes, obesity, skin infections. However, none of references relate to the use of macroalgae extract or composition for enhancement of sexual health.

An alternative natural source of supplement for sex enhancement in the form of a macroalgae composition is described herein, which is obtained from solvent extraction of macroalgae, which in general terms is also called seaweed, occurring as various genera of green, red, and/or brown seaweeds. It is also found that, macroalgae compositions herein are comprised of a specific ratio of saturated fatty acids and unsaturated fatty acids and also exhibit effect on various sexual health biomarkers and thus can be useful method for improvement of sexual health.

Sea-weeds are being-utilized as a raw material in functional foods and it is known to extract bioactive constituents from seaweed which can then be used in the preparation of functional foods. However, the extraction methods known heretofore typically require a long extraction time, use solvents that are generally toxic and inflammable, and need a very high temperature and/or pressure.

As reported herein, exhaustive trials have been carried out for devising a method for improvement of sexual health by employing composition prepared from a natural source such as macroalgae. The composition is comprised of specific ratio, for example an area percentage ratio, of saturated fatty acids and unsaturated fatty acids being at or about 1:0.5 to at or about 1:10 and were evaluated for theireffect on various biomarkers related to sexual health. It was observed that the composition consistently exhibit either desired enhancement and/or the inhibition of certain biomarkers, thus suggesting overall improvement in sexual health. The process for preparation of macroalgae composition is convenient and economic with respect to minimum extraction time, use of low reaction temperature and pressure and employs safe solvents for extraction, so that the composition is safe for human consumption.

Thus, methods herein improve sexual health in subjects in need thereof, by administering a macroalgae composition in an effective daily dose. The composition used herein is a safe alternative natural source for improving sexual health which is prepared by an economically viable process, making use of available commercial equipment and is comprised of a specific ratio, for example an area percentage ratio, of saturated to unsaturated fatty acids at or about 1:0.5 to at or about 1:10.

In an embodiment, methods for improvement of sexual health and macroalgae compositions used therein are described herein.

In an embodiment, macroalgae compositions herein are comprised of a macroalgae extract, which can be administered either as such or can be suspended in a suitable oil medium. The extract can be also formulated in the form of beadlets or spray dried powders and can be compressed in tablets or filled in capsules. For formulations into other types of finished dosage forms, a variety of excipients can be used such as binder, filler, disintegrant, diluents, carrier, glidant.

The fatty acids extracted from the macroalgae do not exist in the desired ratios as stated herein, but where the macroalgae is extracted as per the preparation methods herein, followed by its derivatization as ester and quantification by gas chromatography. This enables quantification in the form of medium chain and long chain fatty acids. Residual solvents, if any, or other components remaining after the extraction do not impact the effectiveness of the extract for the composition.

In an embodiment, a method is provided for improving sexual health in a subject in need of such improvement in sexual health by administering a macroalgae composition in an effective daily dose.

In an embodiment, a method is provided for improving sexual health by administering a macroalgae composition including a macroalgae extract, to a subject in need thereof.

In an embodiment, a method is provided for improving sexual health by using a macroalgae composition and evaluating its effect(s) on health parameters and/or associated biomarkers related to sexual health.

In an embodiment, a method is provided for improving sexual health by administering a macroalgae composition including a macroalgae extract, comprising a specific ratio, for example an area percentage ratio, of saturated fatty acid and unsaturated fatty acid, which is at or about 1:0.5 to at or about 1:10.

In an embodiment, compositions used herein include a macroalgae extract, comprising saturated fatty acids such as palmitic acid, stearic acid, lauric acid, myristic acid, heptadecanoic acid, aratidic acid, behenic acid, and the like, or combinations thereof.

In an embodiment, a macroalgae extract is comprised of unsaturated fatty acids selected from the group of oleic acid, linoleic acid and linolenic acid, palmitoleic acid, ecasanoic acid, erucic acid, and the like or the combination thereof.

In an embodiment, a method is provided for improving sexual health by administering a macroalgae composition including a macroalgae extract, wherein the macroalgae is selected from the group of, but not limited to green, red, brown seaweeds.

In an embodiment, the macroalgae is selected from various genera of brown seaweeds such as Ascophyllum, Fucus, Furcelleria, Laminaria and the like, or combinations thereof. More particularly, the composition may be comprised of the macroalgae extract obtained from species of brown seaweeds such as Ascophyllum nodosum, Fucus vesiculosus and other species of Furcelleria and Laminaria.

In an embodiment, compositions herein are prepared by an industrially viable process by employing non-polar, semi-polar, polar solvents or combinations thereof.

In an embodiment, macroalgae compositions herein are prepared by industrially viable process, employing organic and/or inorganic solvents, and evaluated for health parameters or biomarkers related to sexual health.

In an embodiment, a method is provided for improvement in sexual health by administering a macroalgae composition to a subject in need thereof in an effective daily dose, and evaluating the effect of the composition by suitable methods.

In an embodiment, a period of daily doses can range for example but not limited to at or about 3 months to at or about 2 years. It will be appreciated that there may be no fixed time period for the daily doses as it may be less or longer than such range.

In an embodiment, a method is provide for improving sexual health by administering a macroalgae composition in an effective daily dose, which enhances testosterone synthesis and nitric oxide synthesis.

In an embodiment, a method is comprised of administering a macroalgae composition to a subject in need thereof, in an effective daily dose of at or about 250 mg to at or about 4000 mg, for improvement in sexual health.

In an embodiment, a method is provided for improving sexual health by administering a macroalgae composition in an effective daily dose, which inhibits phosphodiesterase 5 and phosphodiesterase 11.

In an embodiment, a method is provided for improvement of sexual health in both sexes by administering a macroalgae composition, comprising a specific ratio of unsaturated and saturated fatty acids, in an effective daily dose to a subject in need thereof, which effectively enhances testosterone and nitric oxide synthesis and inhibits phosphodiesterase 5 and phosphodiesterase 11. The compositions used herein are prepared by industrially viable and economic processes and include a macroalgae extract comprising saturated and unsaturated fatty acids in ratio of at or about 1:0.5 to at or about 1:10.

Methods are described for improving sexual health by administering a macroalgae composition in an effective amount to the subject in need of such improvement. The methods described herein are comprised of administering a macroalgae composition including a macroalgae extract, comprised of a specific ratio, for example an area percentage ratio, of saturated and unsaturated fatty acidswhich is at or about 1:0.5 to at or about 1:10, to a subject in an effective daily dose, and evaluating the effects on sexual health related biomarkers.

Compositions used herein include a macroalgae extract prepared by solvent extraction of macroalgae selected from green, red and/or brown seaweeds or the combinations thereof. A process is described for preparation of macroalgae composition, which is convenient, economic, does not use high temperature or pressure and employs safe solvents for extraction, so that the resulting composition is safe for human consumption. The extract is prepared by treating green, brown or red seaweeds with suitable non-polar, semi-polar and polar organic and/or inorganic solvents and the combinations thereof.

The method as described herein is comprised of administering a macroalgae composition to a subject in need thereof in an effective daily dose for improvement of sexual health. The compositions used herein can be administered to the subject in need thereof in an effective daily dose of at or about 250 mg to at or about 4000 mg of the active material in the extract, for improving sexual health in both the sexes.

Macroalgae compositions herein include a macroalgae extract, comprised of unsaturated and saturated fatty acids in a specific ratio and are useful for improving sexual health, when used in an effective amount. The method described herein is comprised of administering a macroalgae composition including a macroalgae extract comprised of saturated fatty acids such as palmitic acid, stearic acid, and the like; and unsaturated fatty acids such as oleic acid, linoleic acid and linolenic acid in specific ratio, which is at or about 1:0.5 to at or about 1:10.

The composition used herein exerts effect on biomarkers related to sexual health such as testosterone and nitric oxide synthesis, wherein both these biomarkers are enhanced by administering a macroalgae composition to a subject in need thereof. The method, as described herein also exerts effect on biomarkers such as phosphodiesterase 5 and 11, wherein both these biomarkers are inhibited, due to administration of the macroalgae composition in an effective amount. The method for improvement in sexual health employs macroalgae composition which is safe for human consumption, prepared by economical and industrially viable processes, which can be administered in effective amounts to the subject in need thereof.

### DETAILED DESCRIPTION

Described herein are methods for improving sexual health by administering a macroalgae composition in an effective daily dose to a subject in need of such improvement, and to evaluating the effect of the composition on health parameters related to sexual health. The compositions used herein include macroalgae extract characterized for chemical constituents such as unsaturated and saturated fatty acids by a chromatography technique in the form of area percentage and are evaluated for health applications such as sexual health improvement, using cell lines and bioassays.

The term “sexual health” as used herein refers to health of both male and female sexes and it relates to a state of physical, emotional, mental and social well-being in relation to sexuality; it is not merely the absence of disease, dysfunction or infirmity. Sexual health is affected in humans due to various reasons such as stress, hormonal imbalance, other disease conditions such as hypertension, hypothyroid, addictions, work burden and such related conditions, thus creating need of treatment for improving sexual health.

The term “subject in need of” as used herein refers to a human being of either sex, that is a male or female who is suffering from sexual well-being, because of any underlying reason, at young, moderate or old age.

The term “evaluating effect on sexual health” as used herein refers to method of administering the composition to the subject in need thereof and checking effect by in vivo or in vitro method, by carrying out study on cell line, on biomarkers through the bioassays or in animal models, whichever method is suitable for such evaluation.

As used herein, the term “macroalgae composition” refers to a product which includes active material obtained by solvent extraction of seaweeds preferably brown, green or red marine algae or seaweeds; the composition thus includes macroalgae extract. The brown, green or red marine algae are selected from the group of, but not limited to genera such as Ascophyllum, Fucus, Laminaria, Furcellaria, Sargassum, Chondrus, Caulerpa, Codium, Gracileria, Macrocystis, Monostroma, Porphyra, Cladophora, Halimeda, Bryopsis, Chaetomorpha and the like or the combinations thereof. Seaweed refers to several species of macroscopic, multicellular, marine algae that are found near the seabed. Macroalgae belong to mainly three classes of seaweeds such as Phaeophyta, Chlorophyta and Rhodophyta and various genus and species belonging to these three classes.

In an embodiment, macroalgae compositions herein are comprised of a macroalgae extract, which can be administered either as such or can be suspended in a suitable oil medium. The extract can be also formulated in the form of beadlets or spray dried powders and can be compressed in tablets or filled in capsules. For formulations into other types of finished dosage forms, a variety of excipients can be used such as binder, filler, disintegrant, diluents, carrier, glidant.

The fatty acids extracted from the macroalgae do not exist in the desired ratios as stated herein, but where the macroalgae is specifically extracted as per the preparation methods herein, followed by its derivatization as ester (esterification) and quantification by gas chromatography so as to obtain the ratio of fatty acids and evaluate them. This enables quantification in the form of medium chain and long chain fatty acids. Residual solvents or other components remaining after the extraction, if any, do not impact the effectiveness of the extract for the composition.

The methods described herein relate to administering a macroalgae composition to a subject in need of improvement in sexual health, and to evaluating the effect on related parameters through bioassays. The parameters can be selected from testosterone synthesis, nitric oxide (NO) synthesis, and/or the effect on phosphodiesterase 5 and 11. It is well known that nitric oxide (NO) pathway is related to erectile function. Nitric oxide mediates relaxation of the vascular smooth muscle of the resistance arteries of the corpus cavernosum to facilitate penile erection. It has been shown that testosterone modulates the nitric oxide pathway by regulating the expression and activity of NO synthase isoforms. NO exerts its relaxing action on corpus cavernosum and penile arteries by activating smooth muscle soluble guanylate cyclase and increasing the intracellular concentration of cGMP. Thus, the reported study herein shows the effects of the macroalgae composition on nitric oxide production. A second messenger system which involve cyclic nucleotides, are phosphodiesterases (PDEs) which are key regulatory enzymes. In this study, the effects of macroalgae composition on testosterone, nitric oxide, phosphodiesterase enzymes and also on female hormones were evaluated.

The composition used herein as per the method includes a macroalgae extract prepared by employing food grade solvents, which are safe for human consumption. The solvents may be non-aqueous or sometimes the combination of solvents with water can be used for the extraction. Macroalgae composition is comprised of biologically active unsaturated and saturated fatty acids, wherein fatty acid is selected from the group of, but not limited to palmitic acid, stearic acid, oleic acid, linoleic acid, linolenic acid, and the like or the combination thereof. The ratio of saturated to unsaturated fatty acid is at or about 1:0.5 to at or about 1:10.

The method described herein, is comprised of administering a macroalgae composition, which includes macroalgae extract comprised of saturated fatty acids and unsaturated fatty acids in a ratio of at or about 1:0.5 to at or about 1:10, and which is useful for improvement in sexual health. The composition is prepared by treating suitable seaweed variety with suitable solvents at specific conditions to form an extract which is rich in fatty acids in specific ranges.

In an embodiment, the ratio above is of saturated to unsaturated fatty acids. The dose proposed is an amount of the total fatty acids ranging from at or about 250 mg to at or about 4000 mg daily. In some embodiments, the amount range is at or about 500 mg to at our about 3000 mg daily.

The period of treatment can be said to be ranging from at or about three months to at or about two years. Table 1 herein provides the categorization of fatty acids identified into saturated and unsaturated fatty acids.

Saturated fatty acids are lauric acid, palmitic acid, myristic acid, heptadecanoic acid, arachidic acid, behenic acid and stearic acid.

Unsaturated acids are oleic acid, linoleic acid, linolenic acid, palmitoleic acid, erucic acid and eicasanoic acid

Macroalgae as described herein are marine algae or seaweeds, which are classified into three broad groups or classes, based on pigmentation and other characteristics, such as green Chlorophyceae, (2) Brown-Phaeophyceae and (3) Red-Rhodophyceae. Macrolagae compositions herein include an extract, obtained from brown, red and green seaweed varieties. Macroalgae compositions as described herein, are preferably prepared by extraction of brown macroalgae selected from the group of, but not limited to, Ascophyllum, Laminaria, Fucus, Furcellaria, Undaria, Himanthalia, Alaria, Saccharina; preferably the brown seaweed species are selected from Furcellaria lumbricalis, Laminaria japonica, Fucus vesiculosus, Ascophyllum nodosum, Laminaria saccharina, Laminaria digitata, Himanthalia elongate, Undaria pinnatifida, Macrocystis pyrifera, Alariafistulosa, Alaria marginata, Fucus evanescens, Laminaria cichorioides, Laminaria digitata, Laminaria hyperboria, Saccharina latissima and the like. Macroalgae are harvested, for example from different Atlantic Canada sources or Norwegian Sea beaches or any other suitable sea shores and are used for the preparation of macroalgae compositions herein.

Macroalgae has been used in European countries as a rich source of carbohydrates, because it contains carragenans, alginates and arabinose. It is also known as an alkaline food and therefore helps in maintaining acid base balance in the body and an effective component in a healthy acid-alkaline diet. It also contains high natural iodine levels and has been used to treat hypothyroidism. It is also useful for the treatment of cancer such as breast, endometrial and ovarian cancers. In addition, macroalgae is known to exert the properties such as antioxidant, anti-inflammatory, anti-tumor, antibiotic, antiviral, and it is known for applications in weight management, cholesterol reduction and cardiovascular complications.

In an embodiment, a method for improvement in sexual health includes administering a macroalgae composition in an effective daily dose to a subject in need thereof, and evaluating the effect on related health parameters such as testosterone synthesis, nitric oxide synthesis, effect on phosphodiesterase 5 and 11.

In an embodiment, compositions as used herein may be obtained by carrying out extraction using organic and/or inorganic solvents and the combinations thereof, which are safe for human consumption. The process for preparation of macroalgae compositions herein is comprised of mixing the powdered plant material with suitable amount of solvent with or without stirring at ambient or elevated temperature, ranging from 25 to 70° C. In an embodiment, the ratio of raw material to solvent may range from 1:2 to 1:20.

In an embodiment, the solvents used in the preparation may be selected from the group of, but not limited to non-polar, semi-polar and/or, polar solvents and the combination thereof. Accordingly, the solvents employed in the process may be selected from the group such as, but not limited to, acetone, hexane, petroleum ether (low boiling), petroleum ether (high boiling), ethyl acetate, isopropyl alcohol, ethanol, dichloromethane, methanol, acetonitrile, water and a mixture thereof, more preferably from acetone, ethanol, hexane, water, either alone or in combination thereof.

In an embodiment, macroalgae compositions herein are characterized for chemical constituents such as fatty acids by a chromatography technique and evaluated for health applications, using cell lines and bioassays, in order to observe the effects on biomarkers related to sexual health. In an embodiment, saturated and unsaturated fatty acids are quantified in terms of area percentage.

In an embodiment, macroalgae compositions are evaluated through cell based assays on Mouse Leydig TM3 cells. Cells are cultured in suitable growth medium, incubated and treated with macroalgae composition. Assay for testosterone and nitric oxide synthesis are carried out in the cell cultures and a level of the respective product is quantified by using specific assay kits. Phosphodiesterase activities are assessed using in-vitro assay techniques. The effects are compared with positive controls.

Macroalgae compositions as used herein include an extract prepared from a variety of seaweeds, by employing economical, industrially viable processes and solvents safe for human consumption, thus resulting in a composition which is useful for applications as improvement in sexual health.

In one embodiment, a composition herein is administered to a subject in an effective amounts, ranging from at or about 250 mg to at or about 4000 mg daily dose of the fatty acids of the extract, as such in the form of extract alone or along with at least one food grade excipient as per a formulation which may be produced. It may be also administered in the dosage form such as powder, granules, beadlets, tablets, capsules, suspensions, solutions for oral consumption or any suitable non-oral dosage forms such as topical patches.

The examples given below are provided to illustrate macroalgae composition, process for preparation and application for improvement in sexual health. While the compositions and methods have been described in terms of illustrative embodiments, certain modifications and equivalents will be apparent to those skilled in the art and are intended to be included within the scope of the compositions and methods herein. The details and advantages of which are explained hereunder in greater detail in relation to non-limiting exemplary illustrations.

### EXAMPLES

### A. Macroalgae Composition Preparation Using Solvent Extraction Process

The examples provide an extraction process for different species of macroalgae, using suitable non-polar, semi-polar and/or polar solvents to obtain macroalgae extract composition.

### Example 1

### Preparation of Macroalgae Composition from Genus Fucus

10 kg of powdered material was weighed and taken in a reactor and about 6-10 volumes of hexane was added and stirred for 3 hours at 60−65° C. In this and the following examples, the volume of solvent used is 6-10 times of the raw material (macroalgae), for example if the powdered material is 10 kg, then the volume of solvent is about 60 to 100 lit. The extract was filtered under vacuum through suitable filtration system. The material was re-extracted two more times using similar extraction conditions as above and the extract was filtered under vacuum.

The filtrates were combined and concentrated in a reactor and distillation was carried out at 50° C. The concentrated extract was evaporated to dryness using a rotary evaporator. This was subjected to further fractionation and purification by known methods. Total yield of the extract is 0.77%.

### Example 2

### Preparation of Macroalgae composition from Fucus

The powdered material was weighed and taken in a reactor and 10 volumes of acetone was added and stirred for 3 hours at 45-50° C. The acetone extract was filtered under vacuum using suitable filtration system. The process of extraction of the above treated material was repeated two more times using the same parameters as above and the filtrate was collected. The filtrates were combined and concentrated in a reactor and distillation was carried out at 40° C. The concentrated extract was evaporated to dryness using a rotary evaporator. This was subjected to further fractionation and purification by known methods. Total yield of acetone extract is 0.98%.

### Example 3

### Preparation of Macroalgae Composition from Genus Ascophyllum

The powdered material was weighed and taken in a reactor and 10 volumes of hexane was added and stirred for 3 hours at 60-65° C. The hexane extract was filtered under vacuum employing suitable filtration system. The material retained on the filter cloth is re-extracted two more times using similar conditions and filtered to get the filtrate. The filtrates obtained from all these three extraction processes were combined and concentrated in a reactor and distillation was carried out at 50° C. The concentrated extract was evaporated to dryness using a rotary evaporator. This was subjected to further fractionation and purification by known methods. Total yield of hexane extract is about 1.74%.

### Example 4

### Preparation of Macroalgae Composition from Furcelleria

The powdered material of Furcelleria genus was weighed and taken in a reactor and 10 volumes of hexane was added and stirred for 3 hours at 60-65° C. The hexane extract was filtered under vacuum through appropriate filtration system. The material retained after filtration was re-extracted two more times using similar conditions and filtered to collect the extract. The filtrates were combined and concentrated in a reactor and distillation was carried out at 50° C. The concentrated extract was evaporated to dryness using a rotary evaporator. This was subjected to further fractionation and purification by known methods. Total yield of hexane extract is about 0.25% to 0.75%.

### Example 5

### Preparation of Macroalgae Composition from Genus Laminaria

Powdered material of genus Laminaria was weighed and taken in a reactor and 10 volumes of hexane was added and stirred at 60-65° C. The hexane extract was filtered under vacuum through suitable filtration system. The retained material was re-extracted two more times and subjected to filtration using same conditions as mentioned for first time extraction. The filtrates were combined and concentrated in a reactor and distillation was carried out at 50° C. The concentrated extract was evaporated to dryness using a rotary evaporator. This was subjected to further fractionation and purification by known methods. Total yield of hexane extract is about 0.3% to 0.9%.

### Example 6

### Process for Macroalgae Composition from Genus Chondrus

Powdered material was taken in a three necked round bottom flask and about 10 volumes of hexane was added and stirred for 3 hours at 60° C. on oil bath. The hexane extract was filtered out under vacuum through appropriate filtration system. The material retained after filtration was re-extracted for two more times under similar condition and filtered out. The filtrates were combined and concentrated at 50° C. under vacuum using a rotary evaporator. This was subjected to further fractionation and purification by known methods. Total yield of hexane extract was 0.32%.

### Example 7

### Process for Macroalgae Composition from Genus Chondrus

The powdered material was taken in a three necked round bottom flask and 10 volumes of acetone was added and stirred for 3 hours at 45° C. on oil bath. The acetone extract was filtered under vacuum using a known filtration system. The material retained after filtration was re-extracted two more times and filtered out under similar conditions as mentioned above. The filtrates obtained from these 3 extractions were combined and concentrated at 45° C. under vacuum using a rotary evaporator. This was subjected to further fractionation and purification by known methods. Total yield of acetone extract was 0.52%.

The macroalgae compositions of the examples were characterized for fatty acid content by Gas Chromatography (GC-FID) technique. The column used was DB-FFAP (30 m×0.25 mm, 0.25 μm). The flow rate was maintained about 2.4 ml/min in Split less mode with Nitrogen as carrier gas and injection volume 1.0 μL. The attenuation was retained around −6, wherein the injector temperature and detector temperature was maintained at 220° C. and 260° C. respectively. The hold time for oven temperature was maintained 2 min for up to temperature 70° C., subsequently increased to 5 minutes for up to 240° C. with rate 5° C./min. The total runtime for analysis was maintained around 41.00 min, with air flow 400 ml/min and hydrogen flow 40 ml/min.

The standard preparation of fatty acids were first injected on gas chromatography and analyzed accordingly. Sample preparation containing about 100-200 mg of macroalgae composition was analyzed in GC using the above parameters. Amount of saturated and unsaturated fatty acids was estimated in the form of area percentage. See results of Table 1. The fatty acids were quantified by gas chromatography and represented by area percentage.

TABLE 1
Analysis data of macroalgae composition with respect to saturated and
unsaturated fatty acids

| | | | Ascophylum | Fucus | Chondrus |
|---|---|---|---|---|---|
| | | | nodosum | vesiculosus | crispus |
| Fatty acid | Laminariales | Furcellaria | Example | Example | Example |
| (% area) | Example 5 | Example 4 | 3 | 1 | 6 |
| Saturated | | | | | |
| fatty acids | | | | | |
| Lauric acid | 2.67 | 2.4 | 0.93 | 0.05 | 0.15 |
| Myristic | 11 | 7.53 | 15.63 | 13.77 | 19.36 |
| acid | | | | | |
| Palmitic | 28.29 | 45.37 | 30.74 | 26.2 | 38.61 |
| acid | | | | | |
| Hepta- | 1.23 | 4.79 | 2.05 | 0.32 | 1.2 |
| decanoic | | | | | |
| acid | | | | | |
| Stearic | 4.09 | 5.73 | 2.1 | 2.68 | 4.56 |
| acid | | | | | |
| Arachidic | . . . | 0.7 | 0.29 | 0.14 | 1.61 |
| acid | | | | | |
| Behenic | . . . | 1.16 | 1.26 | 4.63 | 0.22 |
| acid | | | | | |
| Total | 47.28 | 67.68 | 53.00 | 47.79 | 65.71 |
| Un- | | | | | |
| saturated | | | | | |
| fatty acids | | | | | |
| Palmitoleic | 3.97 | 9.34 | 2.35 | 2.45 | 1.33 |
| acid | | | | | |
| Oleic acid | 16.82 | 9.21 | 32.23 | 33.04 | 11.48 |
| Linoleic | 28.01 | 3.14 | 8.68 | 8.73 | 1.57 |
| acid | | | | | |
| Linolenic | 3.92 | 0.71 | 1.32 | 2.75 | 0.06 |
| acid | | | | | |
| Eicasanoic | . . . | 0.95 | 0.01 | 0.55 | 4.5 |
| acid | | | | | |
| Erucic acid | . . . | . . . | . . . | 2.54 | 0.27 |
| Total | 52.72 | 23.35 | 44.59 | 50.06 | 19.21 |
| Ratio of | 1:0.89 | 1:2.89 | 1:1.18 | 1:0.95 | 1:3.42 |
| saturated | | | | | |
| fatty acid: | | | | | |
| unsaturated | | | | | |
| fatty acid | | | | | |

### D. In Vitro Evaluation of Macroalgae Composition on Sexual Health Platform

The effects of macroalgae composition on Leydig cell steroidogenesis was evaluated by measuring testosterone synthesis.

The effect of select test inputs was assessed using the following assay:

i) Testosterone Assay:

The effects of macroalgae composition on testosterone are assessed using a cell based assay. Mouse Leydig TM3 cells (ATCC TMS CRL-1714) were cultured in medium with a 1:1 mixture of Ham's F12 medium and Dulbecco's Modified Eagle's Medium (DMEM), supplemented with 5% horse serum, 2.5% fetal bovine serum, 100 IU/mL penicillin and 100 ug/ml streptomycin for 48 h. The cells were then incubated for 24 hours with advanced DMEM/F12 medium (Gibco Life Technologies #12634-010), supplemented with 1% Horse serum, 0.5% FBS, 1% Pen/Strep, and 1% L-Glutamine. Cells were then treated for 24 h with macroalgae composition at 100 ug/ml. Media was then collected and testosterone levels are quantified using ELISA Kit (Enzo life sciences, Catalog #ADI-900-065). Testosterone levels of treated cells were compared to non-treated cells.

ii) Nitric Oxide Assay:

TM3 cells were cultured following the same protocol than testosterone. Cells were treated for 24 h with macroalgae composition (100 ug/ml) and media is collected. Nitric oxide assay was performed using Nitrate/Nitrite Colorimetric Assay Kit (Caymen #780001). Fucus, Furcelleria and Furcelleria and Laminaria extracts were tested for effect on cells to check effect on biomarkers as shown in the Table 2 below.

iii) Phosphodiesterase V and 11 Assays:

Phosphodiesterase-V and 11 activities have been assessed using in vitro PDE-Glo™ Phosphodiesterase Assay (Promega # PR-V1361), It is a luminescent, high-throughput screening (HTS) method for measuring cyclic nucleotide phosphodiesterase activity.

Human Phosphodiesterase V A1, (sigma Adrich #E9034) or Phosphodiesterase 11A4 (SRP0278), were added to increasing concentrations of macroalgae composition in the presence of the substrate cGMP for 15 min. Then the reaction is stopped using 3-Isobutyl-1-methylxanthine and detected using luciferase-based Kinase-Glo® Reagent. The concentrations that induced 50% inhibition (IC50) of the extracts were calculated.

Results:

Macroalgae compositions (in accordance with the ratio of saturated and unsaturated fatty acids from Table 1) significantly affected all four biomarkers testosterone, nitric oxide and PDE-5 and PDE-11. These extracts enhanced testosterone and nitric oxide synthesis and inhibited phosphosdiesterase 5 and 11, as compared to positive controls such as Fenugreek and Sildenafil.

TABLE 2

| | % | % nitric | | PDE-11 |
|---|---|---|---|---|
| Macroalgae | testosterone | oxide | PDE-5 | IC50 |
| composition | increase | increase | IC50(μg/ml) | (μg/ml) |
| Fucus Extract | 18.5 | 37.34 | 19.50 | 69 |
| Laminaria + | 21.32 | 24.49 | 4.1 | 8.8 |
| Furcellaria | | | | |
| Extract | | | | |
| Laminariales | 22.18 | 78 | 9.5 | 8.9 |
| Extract | | | | |
| Fenugreek | 14.31 | 15.48 | 207.6 | 148 |
| Sildenafil | | | 0.005 | |
| Positive | | | | |
| control for | | | | |
| PDE5 | | | | |
| Tadalafil | | | | 0.01 |
| Positive | | | | |
| control for | | | | |
| PDE-11 | | | | |

We claim:

1. A method for improving sexual health comprising:
administering to a subject in need of improved sexual health, an effective amount of macroalgae composition including a macroalgae extract comprising saturated and unsaturated fatty acids in a ratio of about 1:0.5 to about 1:10, and evaluating the effect on sexual health;
wherein said composition is administered at a dose of between 250 mg per day to 4000 mg per day.

2. The method of claim 1, wherein the macroalgae extract is comprised of saturated fatty acids selected from the group of palmitic acid, stearic acid, lauric acid, myristic acid, heptadecanoic acid, aratidic acid, behenic acid and combinations thereof.

3. The method of claim 1, wherein the macroalgae extract is comprised of unsaturated fatty acids selected from the group of oleic acid, linoleic acid and linolenic acid, palmitoleic acid, ecasanoic acid, erucic acid and t combinations thereof.

4. The method of claim 1, wherein the evaluating includes checking the effect of macroalgae composition on biomarkers related to sexual health.

5. The method of claim 4, wherein the biomarkers are selected from the group of testosterone synthesis, nitric oxide synthesis, phosphodiesterase 5, phosphodiesterase 11.

6. The method of claim 5, wherein the administering enhances testosterone synthesis and nitric oxide synthesis.

7. The method of claim 5, wherein the administering inhibits phosphodiesterase 5 and phosphodiesterase 11.

8. The method of claim 1, wherein the effective amount of macroalgae composition is an amount of the macroalgae extract which is obtained by solvent extraction from brown, red or green macroalgae, selected from species of Ascophyllum, Fucus, Laminaria, Furcellaria, Sargassum, Chondrus, Caulerpa, Codium, Gracileria, Macrocystis, Monostroma, Porphyra, Cladophora, Halimeda, Bryopsis, Chaetomorpha and combinations thereof.

9. A process for preparation of macroalgae composition including an effective amount of macroalgae extract; comprising:
a) treating suitable powdered macroalgae with about 6 to 12 volumes of a solvent for about 1-4 hours at 40-60° C.;
b) filtering out extracted material to separate an extract from step a);
c) re-treating the filtered material from step b), with polar, semi-polar or non-polar solvent at least two more times for 1-4 hours and filtering out the extract;
d) combining the extracts obtained from step c), and concentrating at 50° C. under vacuum to obtain the effective amount of macroalgae extract.

10. The process of claim 9, wherein the solvent is selected from the group of petroleum ether (low boiling), petroleum ether (high boiling), hexane, ethanol, methanol, isopropyl alcohol, n-butanol, acetone, acetonitrile and the mixtures thereof.
